(19) United States
(12) Patent Application Publication
(10) Pub. No.: US 2012/0035667 A1
(43) Pub. Date: Feb. 9, 2012

(54) LOCKING MECHANISMS FOR PIVOTING BONE ANCHORS

(75) Inventors: Matthews Van Nortwick, Memphis, TN (US); Douglas D. Kave, Byhalia, MS (US); Douglas N. Baker, Collierville, TN (US)

(73) Assignee: WARSAW ORTHOPEDIC, INC., Warsaw, IN (US)

(21) Appl. No.: 12/851,714

(22) Filed: Aug. 6, 2010

(51) Int. Cl. A61B 17/86

(52) U.S. Cl. 606/305

(57) ABSTRACT

Bone anchors are provided that include a distal shaft for engaging bony structure of a vertebral body and a proximal post pivotably mounted at or near the proximal end of the shaft during implantation of the bone anchor. The bone anchors include a locking member that is engageable to the post and to the shaft to lock the post in a selected angular orientation relative to the shaft.

### BACKGROUND

The present invention relates to a spinal implant and a manner of using the same, and more particularly, but not exclusively, relates an orthopedic device for treatment of spinal deformities.

The use of spinal implants to address orthopedic injuries and ailments has become commonplace in spinal surgery. In this arena, it is often desired to decrease the invasiveness of the procedures, improve implant integrity, reduce the potential for revision surgery, and provide more positive patient outcomes. Some of these implants utilize bone anchors that depend on interconnection between various components of the bone anchor to assemble the implant and implant systems associate therewith. In certain situations, it may be desirable for the bone anchor to include multi-axial capabilities so that a proximal portion of the bone anchor can pivot relative to the bone engaging portion of the bone anchor to facilitate assembly of the implant and alignment of the vertebrae to which the implant is attached. However, certain types of bone anchors lack a locking capability to prevent pivoting of the bone anchor after the construct is finally assembled and positioned in the patient. Thus, there is a need for additional contributions in this area of technology

### SUMMARY

According to one aspect, a unique bone anchor is provided to engage an elongate connecting element that extends along the spinal column to aid in the assembly of a spinal fixation system. The bone anchor includes multi-axial capabilities during implantation of the connecting element, and is lockable to a fixed orientation during assembly of the fixation system. Other aspects include unique methods, systems, devices, instrumentation, and apparatus involving a bone anchor with multi-axial capabilities that is lockable to a fixed or substantially fixed orientation.

The bone anchors include a distal shaft for engaging bony structure of a vertebral body and a proximal post pivotably mounted at or near the proximal end of the shaft. The bone anchors include a locking member that is engageable to the post and to the shaft to lock the post in a selected angular orientation relative to the shaft.

According to one embodiment, the shaft includes a head about which the distal end of the post is pivotably mounted. The locking member includes a stem portion positioned in a central lumen of the post and a securing portion engageable to the proximal end of the post to contact the proximal end of the stem in the lumen and force the distal end of the stem into contact with the proximal end of the head of the shaft to lock the post in position relative to the shaft. In another embodiment, the post includes a distal end received in and pivotally mounted to a proximal end of the shaft. The locking member includes an elongated stem received in a lumen of the post that opens at a distal end of the post in the shaft and at an opposite proximal end of the post. A proximal securing portion of the locking member engages the post to force the distal end of the stem in contact with a proximally facing surface of the shaft. In yet another embodiment, the proximal end of the shaft forms a receptacle that pivotably houses a ball-shaped member at a distal end of the post. The locking member includes an elongated stem received in a lumen of the post. The lumen opens at a distal end of the ball-shaped member in the receptacle and at an opposite proximal end of the post. A proximal securing portion of the locking member engages the post to force the distal end of the stem in contact with an inner surface of the receptacle. In a further refinement of this embodiment, the ball-shaped member is slotted so that the stem of the locking member expands the ball-shaped member to frictionally engage the inner surface of the receptacle. In a further refinement of any embodiment, an insert is provided in the lumen of the post that is deformed by the stem of the locking member into contact with the shaft to lock the post in position relative to the shaft.

Related features, aspects, embodiments, objects and advantages of the present invention will be apparent from the following description.

### BRIEF DESCRIPTION OF THE DRAWINGS

FIG. 1 is a posterior view of a spinal fixation system engaged to the spinal column of a patient.

FIG. 2 is an elevation view of a bone anchor of the spinal fixation system of FIG. 1.

FIG. 3 is an end elevation view of the bone anchor of FIG. 2.

FIG. 4 is an enlarged detailed view of a portion of the bone anchor of FIG. 2.

FIG. 5 a perspective view of a locking member of the bone anchor of FIG. 2.

FIG. 6 is a longitudinal cross-section of a portion of the bone anchor of FIG. 2.

FIG. 7 is a perspective view of another embodiment bone anchor.

FIG. 8 is an elevation view in partial longitudinal cross-section of the bone anchor embodiment of FIG. 7.

FIG. 9 is a perspective view of another embodiment bone anchor.

FIG. 10 is a cross-section view along line 10-10 of FIG. 9.

FIG. 11 is an elevation view in partial longitudinal cross-section along line 11-11 of the bone anchor embodiment of FIG. 9.

FIG. 12 is an elevation view of a post of the bone anchor of FIG. 9.

FIG. 13 is a longitudinal cross-section of the post of FIG. 12.

FIG. 14 is a perspective view of another embodiment bone anchor.

FIG. 15 is an elevation view in partial longitudinal cross-section along line 15-15 of the bone anchor embodiment of FIG. 14.

FIG. 16 is a perspective view of another embodiment bone anchor.

FIG. 17 is a longitudinal section view of the bone anchor of FIG. 16.

FIG. 18 is an elevation view of the bone anchor of FIG. 16.

FIG. 19 is an elevation view of the bone anchor of FIG. 16 rotated 90 degrees about its central longitudinal axis from the orientation of FIG. 18.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

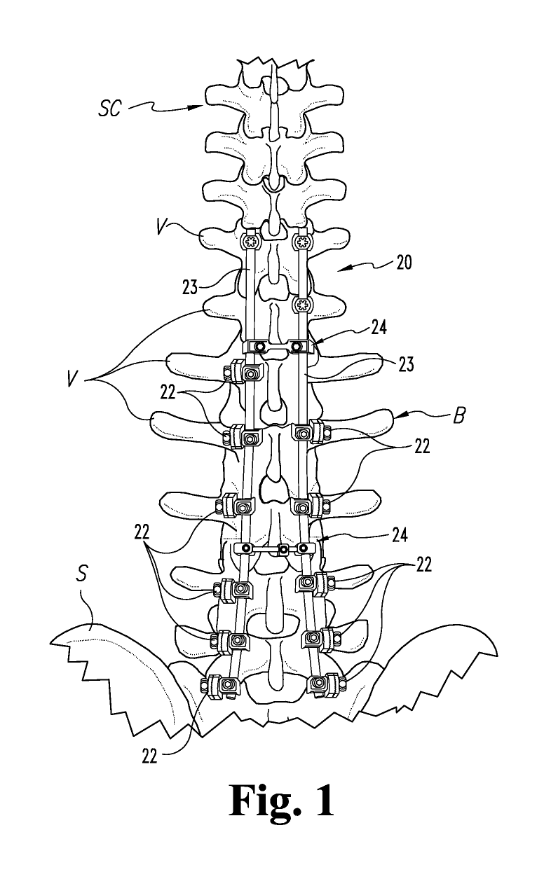

FIG. 1 illustrates a posterior spinal stabilization system 20 implanted at a desired skeletal location of a patient. More specifically, as depicted in FIG. 1, system 20 is affixed to one or more of bones B of the spinal column segment SC from a posterior approach. Bones B include one or more vertebrae V and sacrum S of spinal column segment SC. Spinal fixation system 20 may be employed in spinal column segments SC including sacrum S and one or more vertebrae V, or in spinal column segments that comprise two or more vertebrae V. System 20 includes several bone attachment devices 22 and at least two elongate spinal connecting elements 23 structured to selectively interconnect with bone attachment devices 22. In system 20, bone attachment devices 22 are affixed to various locations of the spinal column segment SC and interconnected with connecting elements 23, which are positioned on opposite sides of the medial or sagittal plane of the spinal column and extend in the cephalad-caudal direction to provide bi-lateral stabilization. Connecting elements 23 may also be interconnected by one or more crosslink devices 24 that extend medially-laterally across the sagittal plane to provide additional stabilization for treating spinal disorders.

Posterior stabilization system 20 may be used for, but is not limited to, treatment of degenerative spondylolisthesis, fracture, dislocation, scoliosis, kyphosis, spinal tumor, and/or a failed previous fusion associated with spinal column segment SC. Furthermore, spinal column segment SC may comprise any one or combination of the cervical, thoracic, lumbar and sacral regions of the spinal column. In certain procedures, spinal stabilization system 20 is secured to a spinal column segment SC with bone attachment devices 22 that include a distal bone engaging portion and a shaft portion that extends proximally from the bone engaging portion. The connecting elements 23 are offset to a side of respective ones of the bone attachment devices 22 and mounted to the proximal shaft portion with a coupling assembly. In one specific embodiment, the coupling assembly is like that found in Medtronic Sofamor Danek's TSRH® 3D spinal system. Of course, any suitable coupling assembly may be used to secure connecting elements 23 to one or more of the bone attachment devices. Furthermore, the bone attachment devices may be employed in surgical procedures and fixation systems like those described in the 2009 Medtronic Sofamor Danek USA, Inc. publication entitled “TSRH-3D PLUS MPA™ SPINAL INSTRUMENTATION DEFORMITY AND DEGENERATIVE SURGICAL TECHNIQUE”, which is incorporated herein by reference.

Connecting elements 23 can be in the form of an elongated spinal rod. The spinal rod may be solid or hollow along some or all of its length and/or may be of homogenous or heterogeneous composition. The spinal rod may also be of uniform cross-section along its entire length, or have a variable cross-section along its length. The spinal rod may include one or more interconnected spinal rod portions that lengthen or adjust in length to accommodate growth of spinal column segment SC over time in the cephalad-caudal directions. The spinal rod can be rigid, flexible, or include one or more flexible portions to permit at least limited spinal motion. Other embodiments of connecting element 23 contemplate any suitable spinal stabilization element positionable along the spinal column, including plates, bars, tethers, wires, cables, cords, inflatable devices, expandable devices, and formed in place devices, for example.

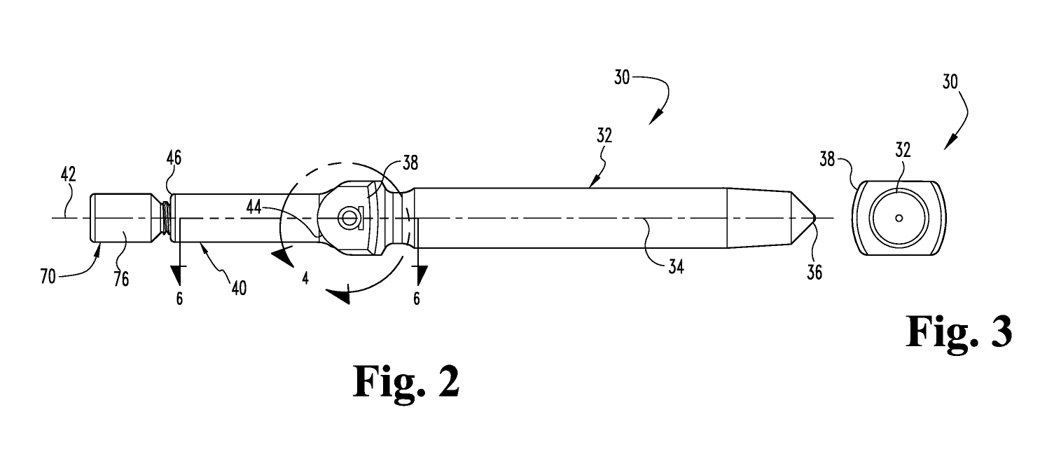

Referring to FIGS. 2-3, there is shown one embodiment of a bone attachment device in the form of bone anchor 30. Bone anchor 30 includes a distal shaft 32 extending on a central longitudinal axis 34 from a distal end 36 to a proximal housing or head 38. Shaft 32 is shown smooth, but may include threads, grooves, flutes, adhesives, wings, gulls, or any other suitable bone engaging structure. Distal end 36 includes a pointed configuration to facilitate entry into bone. A post 40 is pivotally mounted to head 38. Post 40 includes an elongated body extending along a central longitudinal axis 42 from a distal end 44 to an opposite proximal end 46.

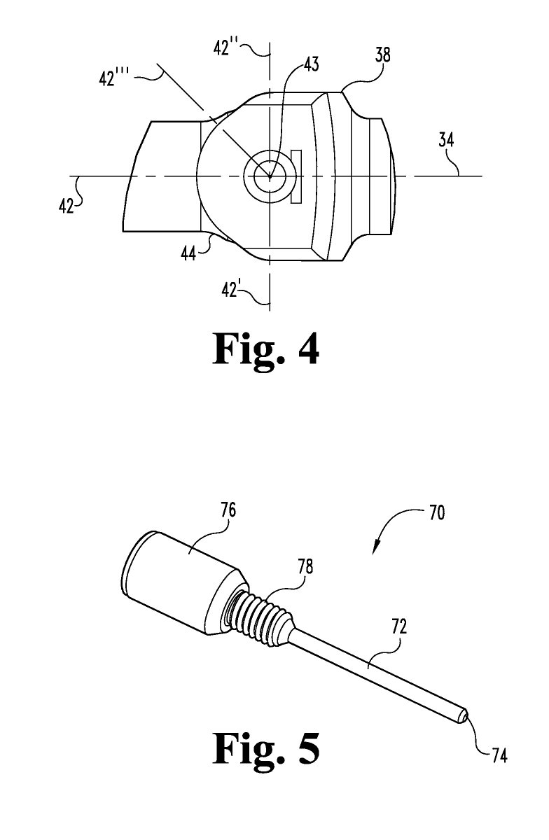

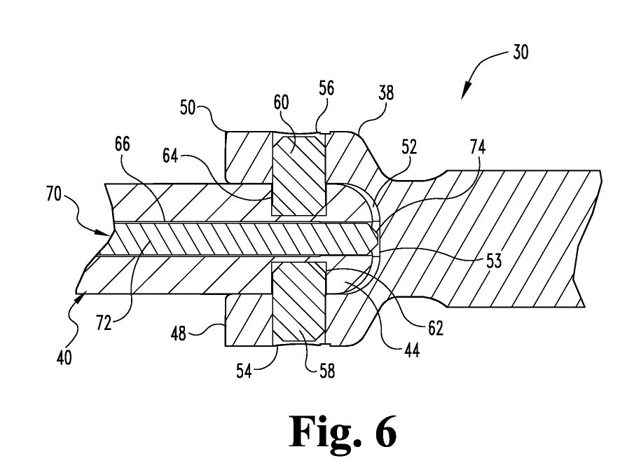

Referring further to FIGS. 4 and 6, head 38 includes a pair of arms 48, 50 extending along opposite sides of a receptacle 52 to form a U-shaped clevis or yoke. Distal end 44 of post 40 is positioned in receptacle 52. Each of the arms 48, 50 includes a bore 54, 56, respectively, that receive respective ones of mounting pins 58, 60. Mounting pins 58, 60 extend from bores 54, 56 and into receptacle 52 for receipt in a respective one of recesses 62, 64 formed in the sides of post 40 adjacent to distal end 44. Pins 58, 60 can be captured by staking a circumferential edge of each in the respective bore 54, 56 of head 38. Post 40 is pivotable about a pivot axis 43 extending through the centers of pins 58, 60. Pivot axis 43 is orthogonal to central longitudinal axes 34, 42 so that central longitudinal axis 42 is positionable at any angle within a single plane that is defined by axes 42, 34. The selected angle of axis 42 relative to axis 34 can range from +80 degrees from central longitudinal axis 34, as shown by the positioning of central longitudinal axis 42′, to −80 degrees from longitudinal axis 34, as shown by the positioning of central longitudinal axis 42″. In a non-pivoted orientation, post 40 is positioned so its central longitudinal axis 42 forms a 180 degree angle with central longitudinal axis 34 so that axes 34, 42 are co-linear with one another. Post 40 is positionable at any angle relative to longitudinal axis 34 between these orientations, as indicated by the positioning of central longitudinal axis 42′″.

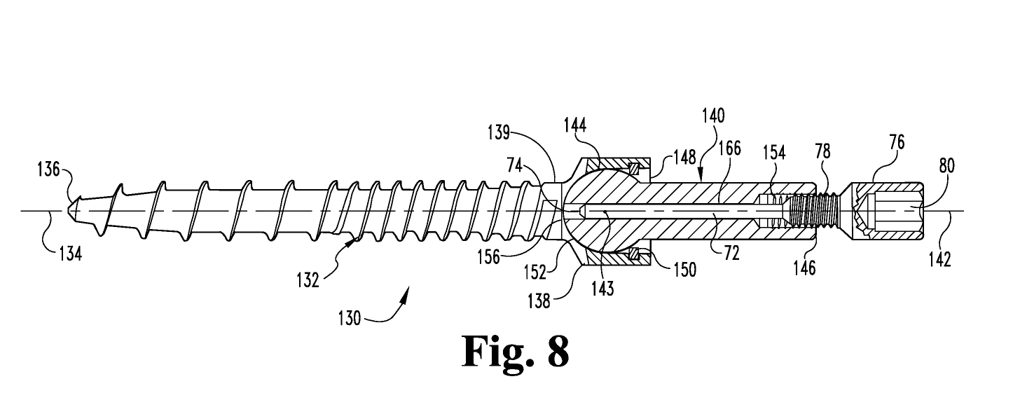

Post 40 includes a central lumen 66 that extends on central longitudinal axis 42 and opens at distal end 44 and at proximal end 46. Central lumen 66 extends between recesses 62, 64 but is isolated from recesses 62, 64 by the body of post 40 in the illustrated embodiment to avoid interference with pins 58, 60. When it is desired to secure post 40 in a desired angular orientation relative to shaft 32, a locking member 70 is positionable through lumen 66 and engaged to post 40 and shaft 32. As further shown in FIG. 5, locking member 70 includes an elongated stem 72 extending from a distal end 74 to a proximal head 76. Locking member 70 also includes a securing portion 78 between head 76 and stem 72 that engages post 40. In the illustrated embodiment, securing portion 78 includes a cylindrical shape with external threads that threadingly engage internal threads at the proximal end opening of lumen 66 (FIG. 8). When it is desired to lock post 40 in a selected angular orientation relative to shaft 32, locking member 70 is threadingly engaged to post 40 and into lumen 66 until distal end 74 contacts shaft 32 in receptacle 52. Receptacle 52 is defined by a bottom surface 53 that extends along and between the inner sides of arms 48, 50. Distal end 74 of locking member 70 creates a point force on shaft 32 that locks post 40 and prevents pivoting movement of post 40 relative to shaft 32.

As shown further below with respect to FIG. 8, head 76 includes an internal driving recess 80 to receive a driving tool to facilitate application of the desired force to locking member 70 in order to lock post 40 in position relative to shaft 32. In the illustrated embodiment, driving recess 80 forms an internal hex-shaped recess. Other embodiments contemplate other configurations for the driving recess, including star-shaped, Allen-shaped, T-shaped, slot-shaped, or other suitable configuration. In still other embodiments, head 76 includes an external configuration to receive a driving tool thereover, including hex shapes, box-shapes, or other suitable external configuration. Head 76 can also be provided with both internal and external configurations to receive driving tools. In yet a further embodiment, the material thickness joining securing portion 78 to head 76 can be sized so that head 76 is severed or removed from the remaining portion of locking member 70 upon application of a threshold torque. This provides a reduced profile or no profile configuration for locking member 70 extending from the proximal end of post 40 when post 40 is locked in position relative to shaft 32.

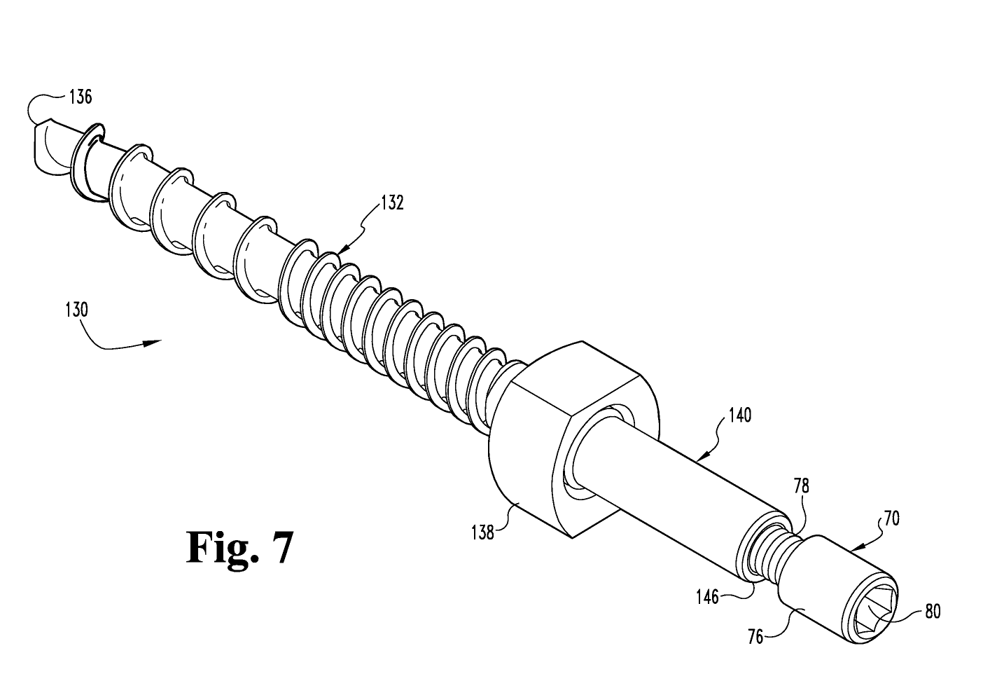

Another embodiment bone attachment device is shown in FIGS. 7-8 in the form of bone anchor 130. Bone anchor 130 is similar to bone anchor 30 and includes a distal shaft 132 extending on central longitudinal axis 134 from distal end 136 to a proximal housing or head 138. Shaft 132 is shown with threads, but may include any other suitable bone engaging structure. Distal end 136 includes a pointed configuration to facilitate entry into bone. Bone anchor 130 further includes a proximal post 140 that is pivotably mounted head 138 of shaft 132 with post 140 extending on a central longitudinal axis 142. Post 140 includes an elongated body extending from a distal ball-shaped member 144 to an opposite proximal end 146. The mounting arrangement of post 140 to shaft 132 allows post 140 to pivot about a point 143 in the central of the ball-shaped member so that central longitudinal axis 134 and central longitudinal axis 142 define multiple planes within which post 140 can pivot relative to shaft 132.

Head 138 extends outwardly from shaft 132 and defines a receptacle 152 that receives ball-shaped member 144 of post 140. Head 138 defines a proximal end opening 148 and a retaining member 150 projecting into opening 148 to capture ball-shaped member 144 in receptacle 152 while allowing post 140 to pivot in any plane to an angular orientation relative to shaft 132. In order to lock post 140 in a selected angular orientation relative to shaft 132, locking member 70 can be positioned with stem 72 in longitudinal lumen 166 of post 140 and securing member 78 is threadingly engaged to internal threads 154 at the proximal end opening of lumen 166. Locking member 70 is advanced until distal end 74 contacts an internal concave surface 156 of receptacle 152 to provide a point force sufficient to lock post 140 in position relative to shaft 132.

In the illustrated embodiment of FIGS. 7-8, shaft 132 is threaded and includes a double-lead thread which is provided with a flat thread crest and angled leading and trailing thread faces. In the illustrated embodiment, one of the leads of the double-lead thread extends along the entire length of the threaded shaft, with the other lead extending along only a proximal portion of the threaded shaft. To provide this type of thread configuration, one of the double-leads is removed from the distal portion of the threaded shaft. In other embodiments, both of the interleaved threads of the double-lead thread extend along substantially the entire length of the threaded shaft. Each lead of the double-lead thread defines a constant and uniform thread pitch. In some embodiments, the threaded shaft defines a uniform major thread diameter which runs out onto a cylindrical portion 139 of the shaft that joins head 138. In other embodiments, the proximal portion of the threaded shaft defines a uniform major thread diameter, but the distal portion of the threaded shaft defines a major thread diameter which inwardly tapers toward the distal tip 136. In other embodiments, the major diameter of the threaded shaft portion is larger than the outer diameter of the cylindrical portion 139. However, in other embodiments, the major diameter of the threaded shaft portion is smaller than the outer diameter of the cylindrical portion 139. Furthermore, in some embodiments, the distal portion of the threaded shaft defines a uniform minor thread diameter extending proximally from the distal tip 136, with a proximal portion of the threaded shaft defining a minor thread diameter which outwardly tapers in a proximal direction. In other embodiments, the distal portion of the threaded shaft defines a minor thread diameter that outwardly tapers from the distal tip 136 in a proximal direction at a first taper angle, with an intermediate portion of the threaded shaft defining a uniform minor thread diameter, and with a proximal portion of the threaded shaft defining a minor thread diameter which outwardly tapers in a proximal direction at a second taper angle which is less than the first taper angle. Additionally, in some embodiments, the distal end portion of the threaded shaft is provided with a pair of oppositely-disposed, semi-circular cutting flutes that intersect at least two full thread turns and which extend to the minor diameter of the thread to provide the threaded shaft with self-cutting and/or self-tapping capabilities.

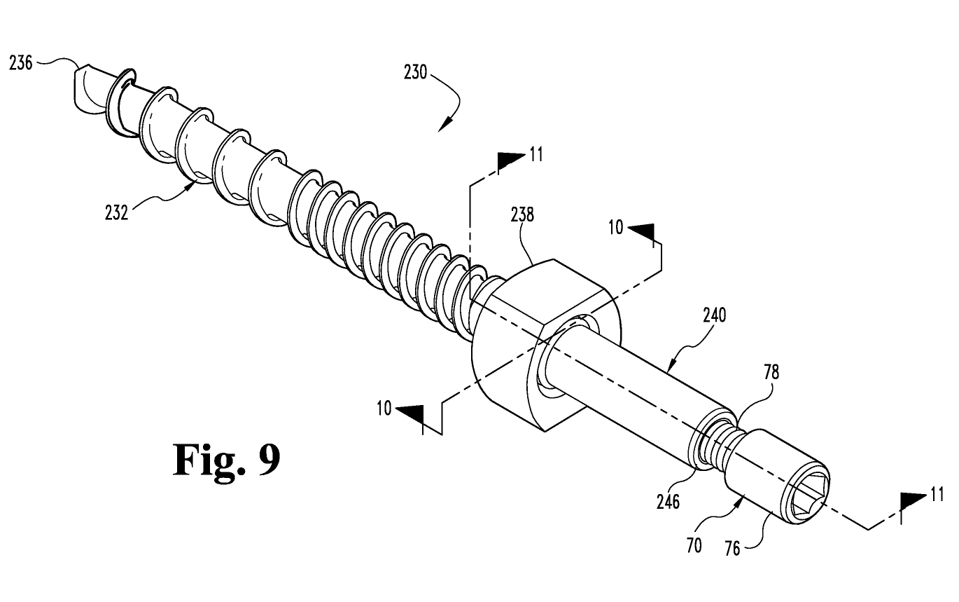

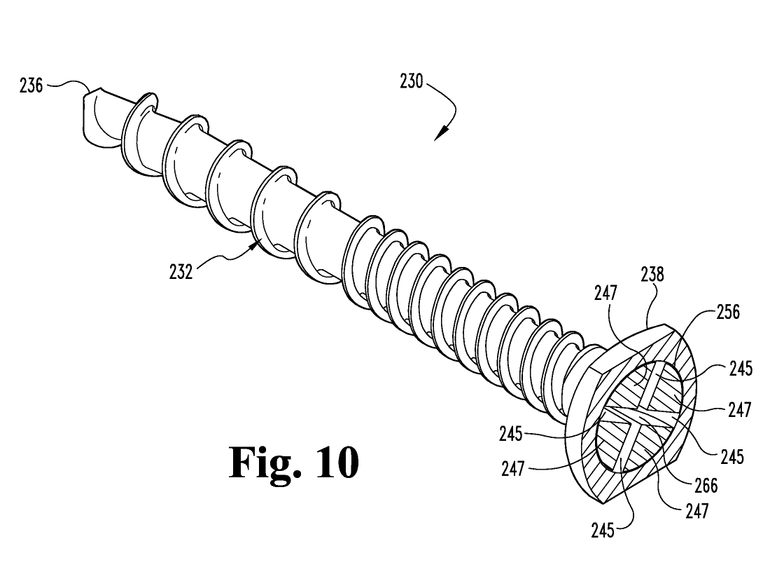

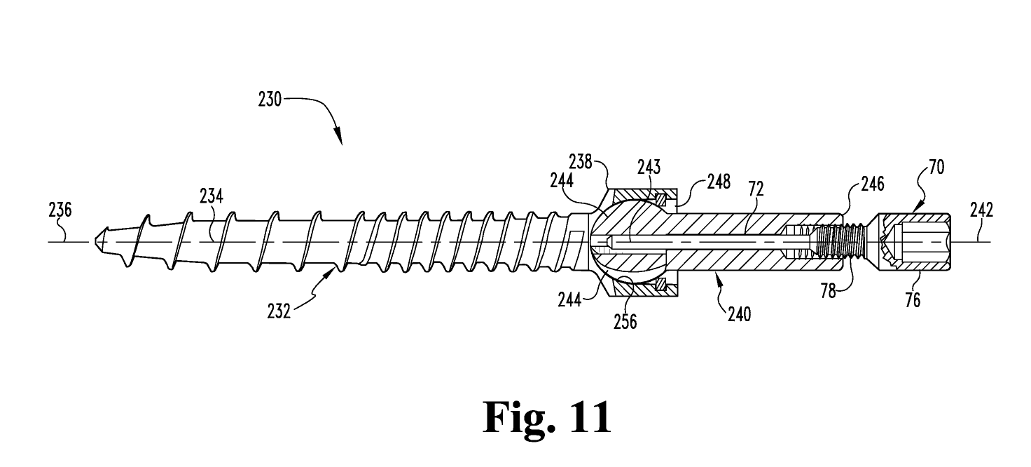

FIGS. 9-13 show another embodiment bone anchor 230 that is similar to bone anchor 130. However, bone anchor 230 includes a post 240 with an expandable ball-shaped end member 244 that expands to lock it in position in head 238 of shaft 232. Shaft 232 includes an elongated body extending on central longitudinal axis 234 from distal end 236 to proximal head 238. Post 240 includes an elongated body extending on central longitudinal axis 242 from ball-shaped member 244 at its distal end to proximal end 246. Ball shaped member 244 pivots about its center 243 in head 238 in any direction relative to central longitudinal axis 234. Locking member 70 is positionable through lumen 266 of post 240 to expand ball-shaped member 244 into engagement with the inner wall surfaces 256 of receptacle 248 of head 238.

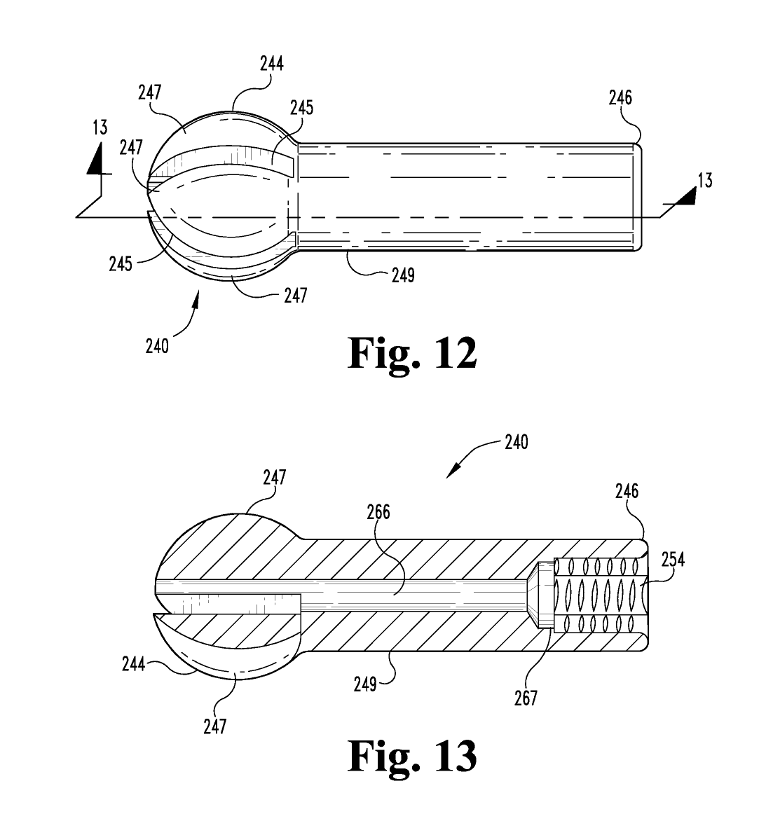

As shown in FIGS. 12-13, post 240 includes a number of slots 245 that bifurcate ball-shaped member 244 into a number of segments 247. Slots 245 open at the distal end of post 140, and extend into ball-shaped member 244 to intersect lumen 266. Slots 245 extend proximally through ball-shaped member 244 to cylindrical sleeve portion 249 of post 240. Sleeve portion 249 extends from ball-shaped member 244 to proximal end 246. Similar to posts 40 and 140, sleeve portion 249 of post 240 includes internal threads 254 formed in an enlarged proximal portion 267 of lumen 266 adjacent to proximal end 246.

In use, shaft 232 is engaged to the bony structure of the vertebra, and post 240 is manipulated to the desired angular orientation relative to shaft 232. Locking member 70 is inserted into lumen 266 of post 240 and securing portion 78 is threadingly engaged to threads 254. Stem 72 acts as a wedge to open ball member 244 by moving segments 247 radially outwardly to frictionally engage inner wall surface 256 of head 238 and provide an interference fit therewith. In addition, the distal end 74 of stem 72 can be advanced to penetrate or seat against the portion of inner wall surface 256 aligned with lumen 266.

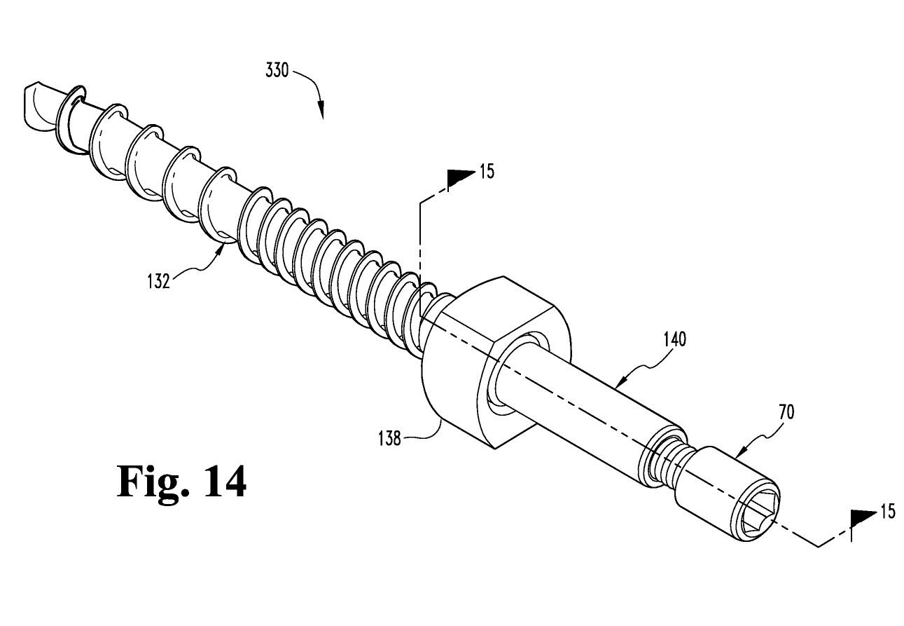

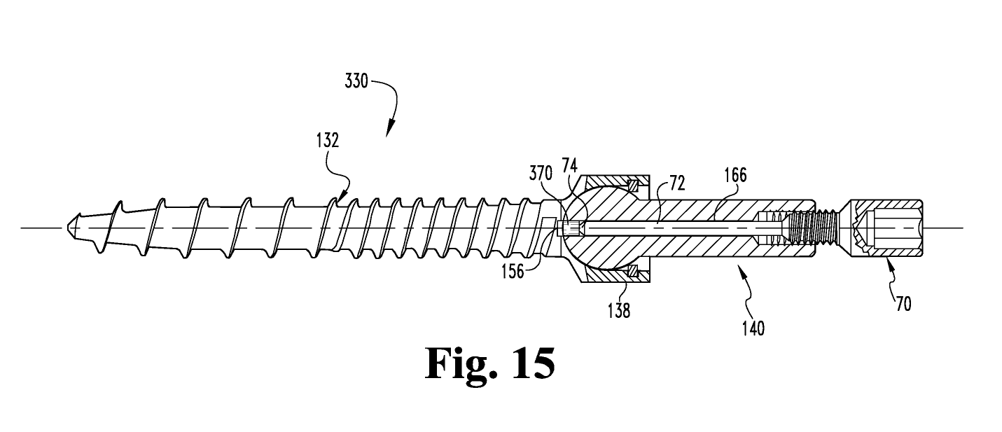

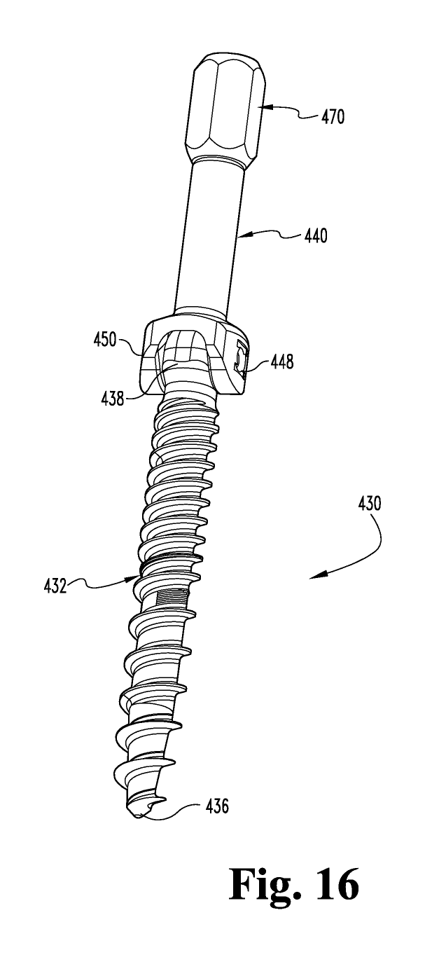

Referring to FIGS. 14-15, there is shown another embodiment bone anchor 330 that is identical to bone anchor 130 discussed above except that a material insert 370 is provided in lumen 166 of post 140. Material insert 370 is pushed distally by distal end 74 of stem 72 as locking member 70 is threadingly engaged in post 140. Material insert 370 can be made from a material that deforms more readily than the material of shaft 132 so that insert 370 deforms as it is compressed between stem 72 and inner surface 156 of head 138 of shaft 132 while reducing the potential for undesired deformation of shaft 132.

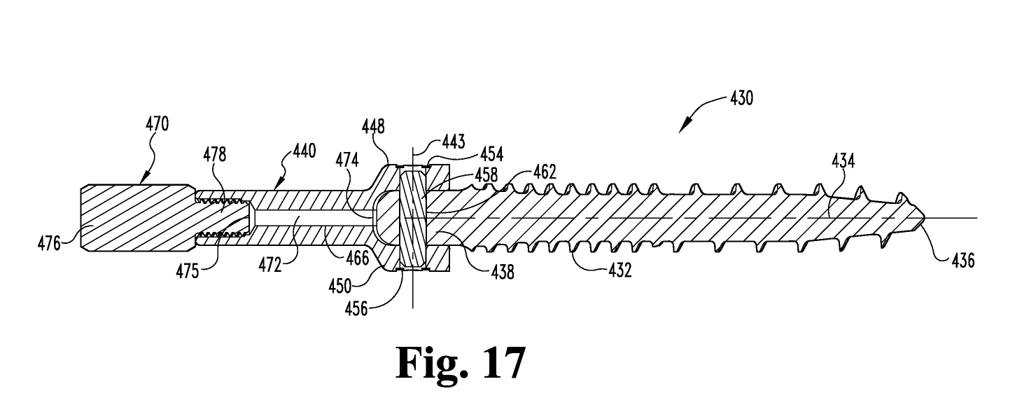

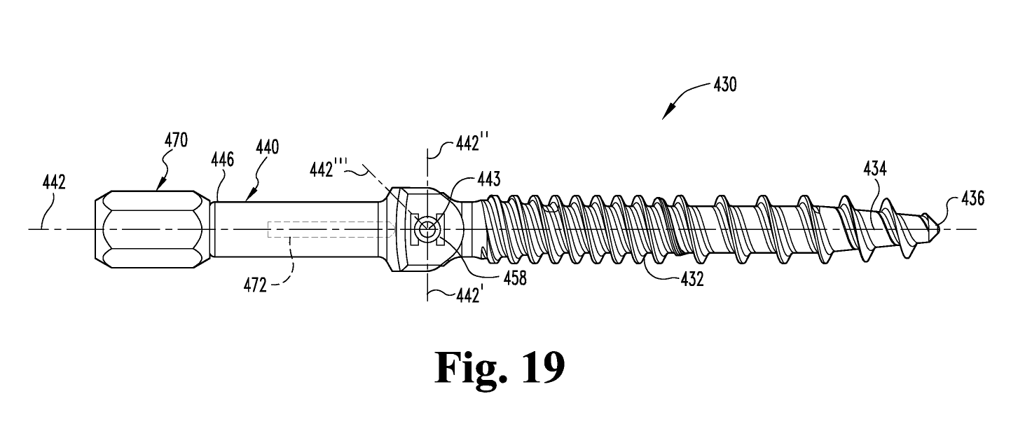

Referring to FIG. 16-19, there is shown another embodiment of the bone attachment device in the form of bone anchor 430. Bone anchor 430 includes a distal shaft 432 extending on a central longitudinal axis 434 from a distal end 436 to a proximal head 438. Shaft 432 includes threads as shown, but may include any other suitable bone engaging structure. Distal end 436 includes a pointed configuration to facilitate entry into bone. A post 440 is pivotally mounted around head 338. Post 440 includes an elongated body extending along a central longitudinal axis 442 from a distal end 444 to an opposite proximal end 446.

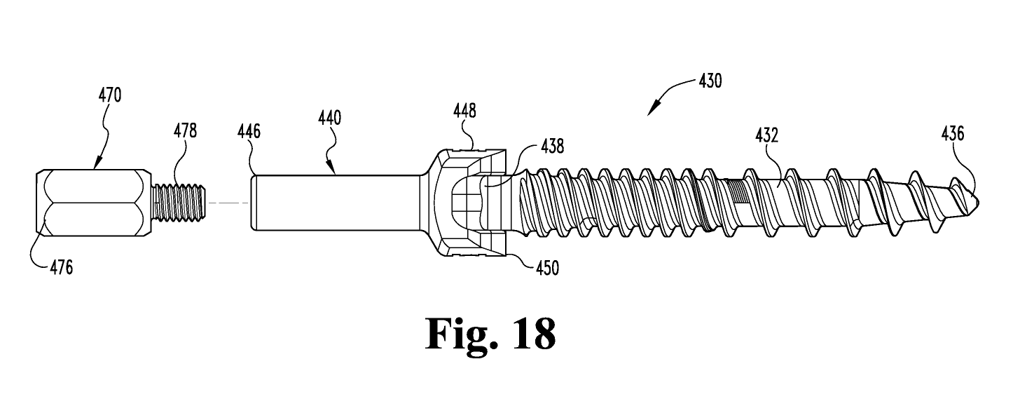

Distal end 444 of post 440 includes a pair of arms 448, 450 extending along opposite sides of head 438 to form a U-shaped clevis or yoke. Head 438 of shaft 432 is positioned in a receptacle defined between arms 448, 450. Each of the arms 448, 450 includes a bore 454, 456, respectively, that receives mounting pin 458. Mounting pin 458 extends from bore 462 that extends laterally through opposite sides of head 438 and into bores 454, 456 of arms 448, 450. Pin 458 can be fixed in bore 462 so that pin 458 rotates in each bore 454, 456 of arms 448, 450. Post 440 is pivotable about a pivot axis 443 defined by pin 458 that is orthogonal to central longitudinal axes 434, 442 so that central longitudinal axis 442 is positionable at any angle in a plane that is defined by axes 442, 434. The selected angular orientation can range from +90 degrees from central longitudinal axis 434, as shown in FIG. 18 by the positioning of central longitudinal axis 442′, to −90 degrees from longitudinal axis 434, as shown by the positioning of central longitudinal axis 442″. In a non-pivoted orientation, post 440 is positioned so its central longitudinal axis 442 forms a 180 degree angle with central longitudinal axis 434 so that axes 434, 442 are co-linear with one another. Post 440 is pivotable relative to shaft 432 so that central longitudinal axis 442 is oriented at any angle relative to longitudinal axis 434 between these orientations, as indicated by the positioning of central longitudinal axis 442′″.

Post 440 includes a central lumen 466 that extends on central longitudinal axis 442 and opens at distal end 444 and at proximal end 446. When it is desired to secure post 440 in a desired orientation relative to shaft 432, a locking member 470 is positionable into the proximal end opening of lumen 466 and engaged to post 440. Locking member 470 includes a proximal head 476 and a distal securing portion that extends into lumen 466 to contact a proximal end 475 of an elongated stem 472 that is housed in lumen 466. Stem 472 extends from a distal end 474 to a proximal end 475. As locking member 470 is threadingly advanced into lumen 466, securing portion 478 contacts proximal end 475 of stem 472 to push distal end 474 of stem 472 against a proximal end of head 438 when it is desired to lock post 440 in a selected angular orientation relative to shaft 432. Distal end 474 creates a point force on head 438 that locks post 440 in the selected angular orientation and prevents pivoting movement of post 440 relative to shaft 432.

Head 476 includes an external driving configuration to receive a driving tool to facilitate application of the desired force to lock post 440 relative to shaft 432. In the illustrated embodiment, the driving configuration forms an external hex-shape. Other embodiments contemplate other configurations for driving locking member 470, including external box shapes and other shapes, and/or internal recesses that are hex-shaped, star-shaped, Allen-shaped, T-shaped, slot-shaped, or other suitable configuration. In yet a further embodiment, the material thickness joining head 476 to securing portion 478 can be sized so that head 476 is severed or removed from securing portion 478 upon application of a threshold torque. This provides a reduced profile or no profile configuration for locking member 470 extending from the proximal end of post 440 when post 440 is located in position relative to shaft 432.

The bone anchors discussed herein can be used in any suitable spinal stabilization system. Surgical instruments can also be mounted to the bone anchors for using in positioning components of the fixation system and in aligning vertebrae of the spinal column. For example, an elongated extension or other instrument can be removably engaged to the proximal end of the post of the bone anchors discussed herein during implantation. The extension can then be removed so that a locking member or portion of the locking member can be engaged to the post to lock the post in the selected angular orientation relative to the shaft.

The bone anchors discussed herein can be provided in a number of sizes and configurations, including varying lengths, diameters and bone screw thread arrangements. The post can include a smooth outer surface, and may also include a tool engaging configuration formed on its inner and/or outer surfaces. The bone anchors discuss herein can be formed of titanium, stainless steel, cobalt-chrome or any other suitable biocompatible metal or non-metal material. The bone anchors include a proximal post and a distal shaft that is threaded or otherwise configured for anchoring within vertebral bone. The proximal post is connected directly to the distal shaft portion, although embodiments where the post is indirectly connected to the shaft are also contemplated. In some embodiments, a housing or head is provided between the post and shaft to provide a location in which to pivotably mount the post to the shaft. In some embodiments, the head or housing is formed integrally with the shaft. Other embodiments contemplate that the head or housing is integrally formed with the post, or is a separate component that is separately attached the post and to the shaft. In any event, the head or housing provides a connection that allows the post to pivot relative to the shaft. Of course, it is understood that the relative size of the components can be modified for the particular vertebra(e) to be instrumented and for the particular location or structure of the vertebrae to which the bone anchor will be engaged.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A bone anchor, comprising:
an elongated shaft extending along a first central longitudinal axis from a distal end to a head at an opposite proximal end of said shaft, said shaft being positonable in bony structure to secure the bone anchor to the bony structure;
a post extending along a second central longitudinal axis from a distal end to an opposite proximal end, said distal end of said post being pivotably mounted to said proximal end of said shaft so that said post is pivotable relative to said shaft to orient said second central longitudinal axis in a selected angular orientation relative to said first longitudinal axis, said post further defining a lumen extending between and opening at said distal and proximal ends of said post; and
a locking member positionable in said lumen of said post to lock said post in said selected angular orientation relative to said shaft, said locking member including a securing portion engageable to said adjacent a proximal end of said post to secure said locking member to said post and an elongated stem in said lumen that is movable to a locking position as said securing portion is engaged to said post, wherein in said locking position said stem locks said post in said selected angular orientation.

2. The bone anchor of claim 1, wherein said post is pivotally mounted to said head with at least one pin that extends between said post and said head along a pivot axis, said pivot axis being orthogonally oriented to said first and second central longitudinal axes.

3. The bone anchor of claim 2, wherein said distal end of said post includes a pair of arms extending along opposite sides of said head and each of said pair of arms defines a bore extending therethrough to receive said pin extending outwardly from opposite sides of said head.

4. The bone anchor of claim 3, wherein said stem of said locking member is housed in said lumen of said post and said securing portion of said locking member is separate from said stem, said securing portion being movable into said lumen to contact a proximal end of said stem to force a distal end of said stem against said head of said shaft to lock said post in said selected angular orientation.

5. The bone anchor of claim 1, wherein said selected angular orientation ranges from a non-pivoted orientation where said second central longitudinal axis is co-linear with said first central longitudinal axis to a pivoted orientation ranging from +90 degrees to −90 degrees from said non-pivoted orientation.

6. The bone anchor of claim 1, wherein said locking member includes a proximal head portion and said securing portion extends between said proximal head portion and said stem, said stem extending to a distal end that is positionable to contact said shaft as said securing portion is threadingly engaged to said post to lock said post in said selected angular orientation

7. The bone anchor of claim 1, wherein:
said head of said shaft includes a pair of arms extending on opposite sides of a receptacle and said distal end of said post is positioned in said receptacle; and
each of said arms includes a bore and a pin in said bore thereof, said pins extending into recesses formed in opposite sides of said post to pivotally couple said post to said shaft.

8. The bone anchor of claim 7, wherein said lumen extends between said recesses through said distal end of said post.

9. The bone anchor of claim 1, wherein said head of said shaft defines a receptacle and said distal end of said post includes a ball-shaped member pivotably captured in said receptacle.

10. The bone anchor of claim 9, wherein said post includes a number of slots extending through said ball-shaped member that open into said lumen of said post, and said locking member expands said ball-shaped member into frictional engagement with an inner surface of said head defining said receptacle when said locking member is in said locking position.

11. The bone anchor of claim 1, further comprising an insert in said lumen of said post and said elongated stem of said locking member includes a distal end that contacts said insert and deforms said insert against a surface of said head that is aligned with said distal end opening of said lumen to lock said post relative to said shaft.

12. A bone anchor, comprising:
an elongated shaft extending along a first central longitudinal axis from a distal end to a head at an opposite proximal end of said shaft, said shaft being positonable in bony structure to secure the bone anchor to the bony structure;
a post extending along a second central longitudinal axis from a distal end to an opposite proximal end, said distal end of said post being pivotably mounted to said proximal end of said shaft so that said post is pivotable relative to said shaft to orient said second central longitudinal axis in a selected angular orientation relative to said first longitudinal axis, said post further defining a lumen extending between and opening at said distal and proximal ends of said post; and
a locking member positionable in said lumen of said post to lock said post in said selected angular orientation relative to said shaft, said locking member including a securing portion engageable adjacent said proximal end of said post to secure said locking member to said post and an elongated stem in said lumen that is movable to a locking position as said securing portion is engaged to said post, wherein said stem of said locking member is housed in said lumen of said post and said securing portion of said locking member is separate from said stem, said securing portion being movable into said lumen to contact a proximal end of said stem to force a distal end of said stem against said shaft to lock said post in said selected angular orientation.

13. The bone anchor of claim 12, wherein said post is pivotally mounted to said head with at least one pin that extends between said post and said head along a pivot axis, said pivot axis being orthogonally oriented to said first and second central longitudinal axes and said distal end of said stem directly engages a proximal surface of said head proximally of said pivot axis to lock said post in said selected angular orientation.

14. The bone anchor of claim 13, wherein said distal end of said post includes a pair of arms extending along opposite sides of said head and each of said pair of arms defines a bore extending therethrough to receive said pin extending outwardly from opposite sides of said head.

15. The bone anchor of claim 12, wherein:
said locking member includes a head extending from a proximal end of said securing portion;
said lumen of said post includes a proximal end portion that is enlarged relative to a remaining portion of said lumen;
said securing portion engages said post in said enlarged proximal end portion; and
said head is removable from said securing portion upon application of a threshold torque when said stem locks said post in said selected angular orientation.

16. A bone anchor, comprising:
an elongated shaft extending along a first central longitudinal axis from a distal end to a head at an opposite proximal end of said shaft, said shaft being positonable in bony structure to secure the bone anchor to the bony structure;
a post extending along a second central longitudinal axis from a distal end to an opposite proximal end, said distal end of said post being pivotably mounted to said proximal end of said shaft so that said post is pivotable relative to said shaft to orient said second central longitudinal axis in a selected angular orientation relative to said first longitudinal axis, said post further defining a lumen extending between and opening at said distal and proximal ends of said post; and
a locking member to lock said post in said selected angular orientation relative to said shaft, said locking member including a securing portion engageable adjacent to a proximal end of said post and an elongated stem in said lumen of said post, said securing portion being operable to advance said stem toward said shaft to lock said post in said selected angular orientation relative to said shaft.

17. The bone anchor of claim 16, wherein said stem includes a distal end that contacts said head of said shaft to lock said post in said selected angular orientation.

18. The bone anchor of claim 16, wherein said locking member includes a material insert in said lumen of said post that is positioned between a distal end of said stem and said head of said shaft, and said stem deforms said material insert against said head of said shaft as said stem is advanced toward said shaft to lock said post in said selected angular orientation.

19. The bone anchor of claim 16, wherein said distal end of said post defines a receptacle that receives said head of said shaft therein.

20. The bone anchor of claim 16, wherein said head of said shaft defines a receptacle that receives said distal end of said post therein.
